(19) United States
(12) Patent Application Publication
(10) Pub. No.: US 2022/0257880 A1
(43) Pub. Date: Aug. 18, 2022

(54) INHALER

(71) Applicant: Inhaler + Limited, London (GB)

(72) Inventor: Eva Petrikova, London (GB)

(21) Appl. No.: 17/622,920

(22) PCT Filed: Jun. 26, 2020
(86) PCT No.: PCT/GB2020/051563

§ 371 Date: Dec. 27, 2021

(30) Foreign Application Priority Data
Jun. 26, 2019 (GB) 1909172.7

(51) Int. Cl. A61M 15/00

(52) U.S. Cl. CPC A61M 15/0016; A61M 15/0021; A61M 2205/18; A61M 15/009; A61M 2205/0216; A61M 15/0088

(57) ABSTRACT

An inhaler housing (101, 201, 301, 401, 501) comprises a locating portion configured to receive an aerosolized medication canister (102, 302, 404, 502). The inhaler housing also comprises an outlet (101b, 301b, 501b) comprising a mouth-piece (101c, 303c, 401c, 501c). At least one flow directing element 101d, (301d), 501d is disposed within the housing. The at least one flow directing element is configured to direct a flow of aerosolized medication toward the outlet. A space enclosed by the housing is configured to reduce a velocity of said aerosolized medication prior to said aerosolized medication reaching the outlet for inhalation by a user. An inhaler (100, 200, 300, 400, 500) comprising a similar housing with at least one flow directing element and an aerosolized medication canister is also disclosed.

### FIELD OF THE INVENTION

The present invention relates to an inhaler, and in particular to an inhaler comprising spacer flow directing element to direct medication towards an outlet of the inhaler.

### BACKGROUND

Asthma is a chronic disease characterised by recurrent attacks of breathlessness and wheezing, which may vary in severity and frequency for different individuals. Symptoms may occur several times in a day or week in affected individuals, and for some people the symptoms become worse during physical activity or at night. 5.4 million people in the UK are currently receiving treatment for asthma, with the NHS spending around £1 billion pounds per year treating and caring for people with asthma. The UK still has some of the highest rates of asthma sufferers in Europe, and on average three people a day die as a result of asthma.

Asthma suffers typically have excess smooth airway muscle. During an asthma attack, contraction of the smooth airway muscle constricts the airway, making breathing difficult or impossible. Inhalers (such as metered-dose inhalers or MDIs) typically provide medication (such as beta adrenergic agonists or beta agonists) that relaxes the smooth airway muscle. This can help to relieve a constricted airway that may occur during an asthma attack.

However, effective administration of inhaler medication is difficult, requiring technique, timing and coordination which can introduce user error. For example, the user is required to inhale and administer the medication simultaneously. To mitigate the effects of incorrect use, a separate device known as a spacer can be used in conjunction with the inhaler. The spacer is connected to the inhaler when the user needs to use the inhaler. The spacer provides a volume of space between the inhaler and the mouth of the user. The use of a spacer can help to increase the effective dose of aerosolized medication actually reaching the lungs of a user of the inhaler. However, typically spacers are large, awkward devices which are impractical for an inhaler user to carry around.

### SUMMARY

According to a first aspect, there is provided an inhaler. The inhaler may comprise a housing enclosing a space (for example, the housing may define or comprise an internal space or “spacer”). The housing may comprise a locating portion configured to receive an aerosolized medication canister. The inhaler may also comprise an aerosolized medication canister. The housing may also comprise an outlet. The outlet may comprise a mouthpiece. The housing may additionally comprise at least one flow directing element disposed within the housing (for example, disposed within the space enclosed by the housing). The at least one flow directing element may be configured to direct a flow of aerosolized medication towards the outlet. The space enclosed by the housing may be configured to reduce a velocity of the aerosolized medication emitted from the canister prior to the aerosolized medication reaching the outlet for inhalation by a user.

Prior to the invention, asthma sufferers and other users of inhalers have typically been recommended to use an inhaler in combination with a separate device known as a spacer. A spacer acts to reduce the velocity of the aerosol cloud emitted from the medication canister. Use of a spacer can in turn reduce the impact of hand-breath (activation-inhalation) coordination problems, filter out larger aerosol particles to increase a respirable fine particle fraction in the aerosol cloud (improving lung deposition of aerosolized medication), reduce oropharyngeal impaction or deposition and associated local side-effects (for example, due to inhaled corticosteroids), and reduce a fraction of swallowed drug and gastrointestinal absorption. In short, a spacer can improve the effectiveness of an inhaler by increasing an effective does of the aerosolized medication.

However, this generally requires the inhaler user to carry two separate devices, the inhaler and the spacer. In order to provide an effective dose of aerosolized medication, the inhaler must be connected to the separate spacer. This is often difficult and time-consuming, and may even be dangerous or life-threating in case of emergency (such as during an asthma attack).

In contrast, the inhaler of the first aspect of the invention comprises an integral spacer in the form of the space enclosed by the housing. This may advantageously require an inhaler user to carry only a single device, improving portability. Having a single device means an effective dose of aerosolized medication may be administered quickly without requiring the user to connect an inhaler to a separate spacer beforehand.

The at least one flow directing element may further increase an effective dose of aerosolized medication by directing a flow of aerosolized medication towards the outlet. This may reduce waste of aerosolized medication, for example by reducing the likelihood of the aerosolized medication settling on an internal surface of the housing.

The outlet and the locating portion (or the canister) may be disposed or located on substantially opposite sides of the inhaler. This may increase or maximise a distance between the outlet and the canister across the space enclosed by the housing, which in turn may maximise a volume of space enclosed by the housing configured to reduce a velocity of the aerosolized medication prior to the aerosolized medication reaching the outlet for inhalation by a user.

At least one flow directing element may be disposed adjacent or near the canister (for example, adjacent or near a location at which aerosolized medication is emitted, for example a nozzle in fluid communication with the canister), or adjacent or near the locating portion of the housing. Alternatively or additionally, at least one flow directing element may be disposed adjacent or near the outlet. The at least one flow directing element disposed adjacent or near the outlet may be part of or may be disposed on (for example, may extend from) the mouthpiece. In an embodiment, at least one flow directing element is disposed adjacent or near the canister or adjacent or near the locating portion, and at least one flow directing element is disposed adjacent or near the locating portion of the housing. The flow directing elements may be disposed on or adjacent opposing internal surfaces of the housing. The at least one flow directing element disposed adjacent or near the canister or adjacent or near the locating portion may be configured to fluidly interact with the at least one flow directing element disposed adjacent or near the outlet. That is, the at least one flow directing element disposed adjacent or near the canister or adjacent or near the locating portion, and the at least one flow directing element disposed adjacent or near the locating portion of the housing, may be configured to form or define a fluid flow path. The at least one flow directing element, or flow directing elements, may form a primary flow path for aerosolized medication through the space enclosed by the housing. The flow directing elements may be substantially parallel to one another e.g. oriented in substantially the same direction as each other within the housing. This may further increase an effective dose of aerosolized medication for the user by reducing or minimising an amount of aerosolized medication that does not reach the outlet.

A or the canister may be receivable within and/or removable from the locating portion of the housing. This may enable the canister to be removed or replaced (for example, to replace an empty canister with a full canister) as and when required.

The inhaler may further comprise a cap configured to cover the canister when it is received in the locating portion. The cap may therefore substantially seal the locating portion. The cap may be configured to be depressed by a user to actuate the canister in order to emit aerosolized medication into the space enclosed by the housing. The cap (for example, an outer surface of the cap) may be substantially flush with an outer surface of the housing when not depressed by a user. The cap may be removable to enable the canister to be removed or replaced (for example, to replace an empty canister with a full canister) as and when required.

The space enclosed by the housing may be or have a fixed volume. The fixed volume may be sufficient to reduce a velocity of aerosolized medication prior to reaching the outlet for inhalation by a user. The fixed volume may be between substantially 100 ml and substantially 150 ml, and optionally may be substantially 120 ml. Such a volume may be an optimal compromise between a volume large enough to increase an effective dose of aerosolized medication without the inhaler being oversized and difficult to carry around or store. A housing having a fixed volume may remove the need for potentially complex mechanisms configured to expand a volume of the housing from a low volume (for example, for transport and storage purposes) to a volume sufficient for reducing a velocity of aerosolized medication, when required. Such mechanisms may be difficult and time-consuming for the user to operate correctly, particularly in an emergency (such as during an asthma attack).

Alternatively, the space enclosed by the housing may be or have an alterable volume. The volume may be alterable between a first volume and a second volume. The first volume may be smaller than the second volume. The first volume may be to minimise an overall volume of the inhaler for storage and transport. The second volume may be to maximise an overall volume of the housing to increase an effective dose of aerosolized medication. The housing may comprise a mechanism configured to alter the volume of the housing between the first volume and the second volume. The mechanism may be one or more of a concertina mechanism, a telescopic mechanism, a hinge mechanism or other suitable mechanism. The second volume may be larger than the first volume. The mechanism may be configured to increase the volume of the housing from the first volume to the second volume. The mechanism may be configured to decrease the volume of the housing from the second volume back to the first volume.

The mouthpiece may be securable or secured to the housing. The mouthpiece may be moveably secured to the housing. For example, the mouthpiece may be hingedly or slideably secured to the housing. The mouthpiece may be moveable between a first position in which the outlet is open, and a second position in which the outlet is closed. The housing may be substantially sealed when the mouthpiece is in the second position. The mouthpiece (for example, an outer surface of the mouthpiece) may be substantially flush with the housing (for example, an outer surface of the housing) of the housing when the mouthpiece is in the second position. This may improve cleanliness of the inhaler by preventing ingress of foreign matter (for example, dust, water etc.) into the space enclosed by the housing when the inhaler is not in use, for example during storage or transport. The benefit of this may be two-fold. The user may not be at risk of infection or illness by inhaling or ingesting foreign matter inadvertently trapped in the space enclosed by the housing. Additionally, the foreign matter may not interfere with (for example, bond to, react with, dilute etc.) aerosolized medication, thereby increasing an effective dose of aerosolized medication for the user. Additionally, no additional parts are needed to cover the mouthpiece or seal the housing (for example, an additional cap), as the interaction of the mouthpiece and the housing performs this role. This again reduces complexity of operation of the inhaler for the user. The housing may comprise a main body and the mouthpiece may be moveably secured to the main body of the housing.

The mouthpiece may comprise a valve located in or in the vicinity of the mouthpiece. The valve may be an inspiratory valve.

The mouthpiece and/or the housing may be configured to facilitate moving the mouthpiece from the second position to the first position. An outer surface of the housing may be shaped e.g. angled or curved and/or comprise a notch, recess, indentation, or other tactile feature to enable a user to easily apply force to move the mouthpiece from the second position to the first position, and vice versa. Additionally or alternatively the mouthpiece may be shaped or have a projection, lip, ridge or the like to enable a user to easily apply force to move the mouthpiece from the second position to the first position, and vice versa.

The at least one flow directing element may be or comprise one or more of a flat surface or plate(s) and a curved surface or plate(s). The at least one flow directing element may be or comprise a wedge or ridge. The at least one flow directing element may extend from an internal surface of the housing. The at least one flow directing element may be arranged at an angle of between substantially 110° and substantially 130°, or substantially 115° and substantially 125°, or substantially 120° relative to an initial direction of flow of aerosolized medication, and/or an angle of between substantially 50° and substantially 70°, or 55° and substantially 65°, or substantially 60° relative to an internal surface of the housing (e.g. a surface substantially parallel to the mouthpiece when in the second position). Alternatively, the at least one flow directing element disposed within the housing is arranged at an angle of between substantially 15° and substantially 35° relative to an internal surface of the housing. Alternatively, the at least one flow directing element disposed within the housing is arranged at an angle of between substantially 20° and substantially 30° relative to an internal surface of the housing, or at substantially 25° relative to an internal surface of the housing.

For a flow directing element comprising a curved surface or plate, the angle of the flow directing element may be or comprise an angle of an end portion of the curved surface or plate (for example, a portion of the curved surface or plate over which aerosolized medication passes last for the at least one flow directing element disposed adjacent or near the canister, or adjacent or near the locating portion, or a portion of the curved surface or plate over which aerosolized medication passes first for the at least one flow directing element disposed adjacent the outlet). The angle of the at least one flow directing element may depend on a distance between the at least one flow directing element and the outlet or between the at least one flow directing element and the canister or the locating portion respectively. The angle of the flow directing elements may depend on a distance between flow directing elements (for example, a lateral distance between flow directing elements across a width of the housing, rather than a direct distance between flow directing elements). A greater distance between the at least one flow directing element and the outlet, or between the at least one flow directing element and the canister or the locating portion, or between flow directing elements, may require a greater angle relative to an initial direction of flow of aerosolized medication, or a smaller angle relative to an internal surface of the housing. The at least one flow directing element may be curved or angled in more than one direction.

The flow directing elements may be substantially similar (for example, may be or comprise a substantially similar shape, size or angle) to one another. Alternatively, the flow directing elements may be different from one another. A distance of the at least one flow directing element may be between substantially 3 mm and substantially 25 mm from the canister or the locating portion, or from the outlet (for example, a lateral distance from the canister or the locating portion, or from the outlet, across a width of the housing). A distance of the at least one flow directing element from the canister or the locating portion, or from the outlet, may be a distance of between substantially 5% and substantially 25% of a total distance between the canister or the locating portion and the outlet (for example, a lateral distance between the canister or the locating portion and the outlet, across a width of the housing).

The housing or the locating portion may comprise an actuator nozzle. The actuator nozzle may be configured to interact with the canister (e.g. an outlet or nozzle of the canister) to emit aerosolized medication into the space enclosed by the housing. The actuator nozzle may be in fluid communication with the canister when the canister is actuated by a user. The actuator nozzle may extend from an internal surface of the housing. The actuator nozzle may extend substantially perpendicularly from an internal surface of the housing. The locating portion of the housing may comprise the actuator nozzle. The actuator nozzle may assist in locating and retaining the canister in the correct position within the housing. The at least one flow directing element may be configured to direct a flow of aerosolized medication emitted from the actuator nozzle towards the outlet.

The canister may be a metered-dose inhaler (MDI) canister. The medication contained within the canister may not be aerosolized when contained within the canister. The medication may need to be emitted from the canister (and may also need to interact with and be emitted from an additional structure such as an actuator nozzle) in order to become aerosolized.

The housing may comprise a plurality of housing portions. The plurality of housing portions may be configured to be removably attachable to and detachable from one another. The plurality of housing portions may be configured to be removably attached to one another via one or more of corresponding male and female engagement features (for example a tongue and a groove on respective housing portions), corresponding screw threads (for example, corresponding internal and external screw threads on respective housing portions), resilient elastic clips, a press fit or friction fit etc. The attachment mechanism(s) may be configured to be overcome using light manual force, without becoming susceptible to accidental opening. This may improve ease of use for the user. The attachment mechanism(s) may substantially seal the housing when the plurality of housing portions are attached to one another. Alternatively, an additional seal may be used to seal the housing when the plurality of housing portions are attached to one another. A housing comprising a plurality of housing portions which are removably attached to one another may enable an internal surface of the housing to be accessed and cleaned easily and regularly. This may improve hygiene, reduce allergies and increase an effective dose of aerosolized medication administered. The housing may remain substantially sealed when the housing portions are attached to one another and the inhaler is not in use. The housing portions may comprise an upper part and a lower part, a first side part and a second side part or a front part and a back part.

An outer shape of the housing may be or comprise a substantially regular shape. The outer shape of the housing may be or comprise a substantially cuboidal or cubic shape. Alternatively, the housing may be or comprise a substantially ovoid, spheroid, teardrop or triangular prism shape. The teardrop or triangular prism shape may be symmetric or asymmetric. The housing may have or comprise a teardrop or triangular shaped cross-section. A width of the teardrop or triangular shape may increase from a location of the canister within the housing to a location of the outlet (or the mouthpiece) on the housing. A teardrop or triangular shaped housing may improve flow of aerosolized medication through the space enclosed by the housing. External edges of the inhaler may be substantially rounded, bevelled or chamfered. External surfaces of the inhaler may be substantially smooth and continuous. This may enable the inhaler to be easily stored and transported, and may also improve ease of use and comfort for the user.

The housing may further comprise a whistle. The whistle may be disposed in a wall of the housing. The whistle may be configured to produce a sound (for example, a high-pitched whistling sound) if, during use, a user inhales too quickly (for example, a flow rate of air and/or aerosolized medication out of the housing exceeds a threshold). If a user inhales too quickly whilst using the device, advantages provided by a spacer can be reduced. A whistle configured to provide feedback on inhalation flow rate can help to regulate user behaviour to maximise an effective dose of aerosolized medication administered.

The housing, or an outer portion, layer or covering of the housing, may be formed of or comprise a polymer material (for example, polypropylene, polycarbonate etc.) An internal surface of the housing may be or comprise an antistatic material. The housing may be or comprise an antistatic material (for example, an antistatic polymer material). Additionally or alternatively, the internal surface of the housing may comprise an antistatic coating. An antistatic material or coating may increase the effective dose of aerosolized medication by preventing the aerosolized medication from becoming stuck to an internal surface of the housing.

An internal surface of the housing may be or comprise an antibacterial or antimicrobial material. The housing may be or comprise an antibacterial or antimicrobial material. Additionally or alternatively, the internal surface of the housing may comprise an antibacterial or antimicrobial coating (for example, a coating comprising silver such as silver particles).

In an embodiment, one or a plurality of expiratory vents or holes are provided in the housing. The expiratory vents or holes may be provided in a surface of the housing. The expiratory vents or holes are preferably provided in the vicinity of or under the mouthpiece. The plurality of expiratory vents or holes may be provided in a regular or irregular pattern. The plurality of expiratory vents or holes may be provided in a plurality of rows Each row may comprise one or a plurality of expiratory vents or holes. The vents or holes may be arranged symmetrically across the surface of the housing. Alternatively the vents or holes may be provided in a circular, oval, elliptical or other pattern or an array or grid e.g. a rectangular, square or other regular pattern.

According to a second aspect, there is provided an inhaler. The inhaler may comprise a housing enclosing a space. The inhaler may comprise an aerosolized medication canister. The housing may comprise a locating portion configured to receive an aerosolized medication canister. The housing may also comprise an outlet. The outlet may comprise a mouthpiece. The housing may comprise at least one flow directing element disposed within the housing (for example, disposed within a space enclosed by the housing). The at least one flow directing element may be configured to direct a flow of aerosolized medication towards the outlet. A space enclosed by the housing may be configured to reduce a velocity of the aerosolized medication prior to the aerosolized medication reaching the outlet for inhalation by a user.

According to a third aspect there is provided a method of using an inhaler housing of the first aspect or using an inhaler of the second aspect. The method may comprise inserting or removing a canister of aerosolized medication and/or moving the mouthpiece between the first and second positions. The method may comprise breathing in through the mouthpiece when in the first position.

The optional features from any aspect may be combined with the features of any other aspect, in any combination. For example, the inhaler of the second aspect may comprise an inhaler housing of the first aspect and any one or more of the features described with reference to the first aspect. Furthermore, the inhaler housing of the first aspect may comprise any of the optional features described with reference to the inhaler of the second aspect. Features may be interchangeable between different aspects and embodiments and may be removed from different aspects and embodiments and may be added to different aspects and embodiments.

Features which are described in the context of separate aspects and embodiments of the invention may be used together and/or be interchangeable wherever possible. Similarly, where features are, for brevity, described in the context of a single embodiment, those features may also be provided separately or in any suitable sub-combination. Features described in connection with the method may have corresponding features definable with respect to the device and use of the device, and these embodiments are specifically envisaged.

### BRIEF DESCRIPTION OF THE DRAWINGS

The invention will now be described by way of example with reference to the accompanying drawings in which:

FIG. 1 shows an inhaler in accordance with an embodiment of the invention;

FIGS. 2A, 2B and 2C show embodiments of a flow directing element in accordance with an embodiment of the invention;

FIG. 3 shows a cap configured to cover a canister of an inhaler in accordance with an embodiment of the invention;

FIGS. 4A, 4B, 4C and 4D show inhaler housings comprising mechanisms to alter a volume of the housings in accordance with an embodiment of the invention;

FIGS. 5A, 5B and 5C show another inhaler in accordance with an embodiment of the invention;

FIG. 6 shows an inhaler having a housing comprising two detachable housing portions;

FIGS. 7A, 7B and 7C show dimensions of an inhaler in accordance with an embodiment of the invention;

FIGS. 8A and 8B show an inhaler in accordance with another embodiment of the invention;

FIGS. 9A and 9B show an alternative embodiment of a flow directing element in accordance with the invention;

FIG. 10 shows an inhaler in accordance with another embodiment of the invention;

FIG. 11 shows an inhaler in accordance with another embodiment of the invention; and

FIG. 12 shows an inhaler in accordance with another embodiment of the invention.

Like or similar/corresponding reference numbers and designations in the various drawings indicate like elements.

### DETAILED DESCRIPTION

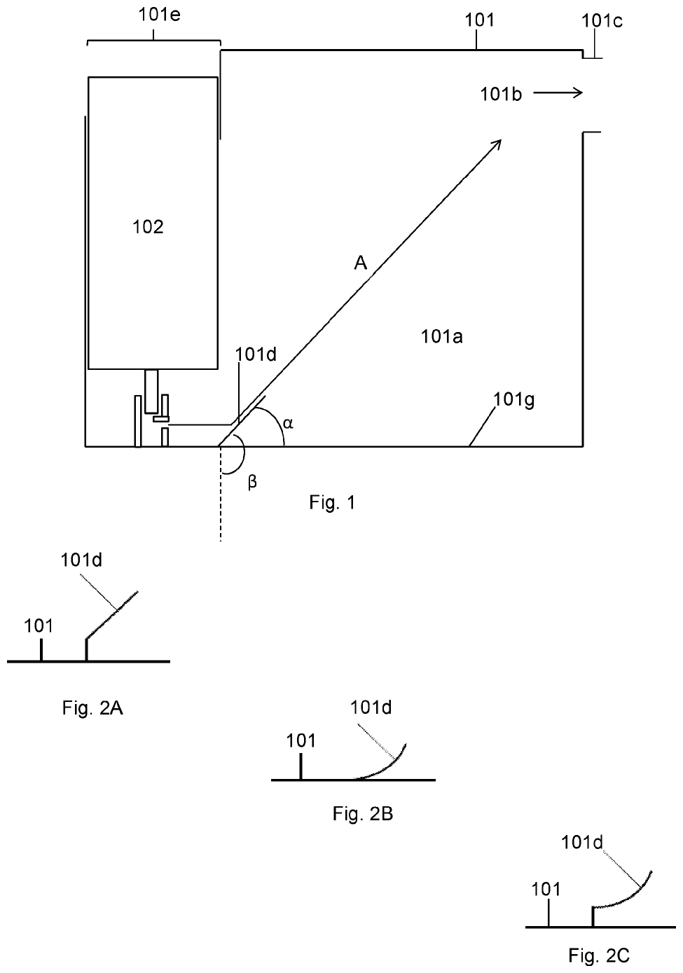

FIG. 1 shows an embodiment of an inhaler 100 in accordance with an embodiment of the invention. The inhaler 100 comprises a housing 101 enclosing a space 101a. The inhaler 100 also comprises an aerosolized medication canister 102. The housing 101 comprises an outlet 101b. The outlet 101b comprises a mouthpiece 101c. The housing 101 also comprises at least one flow directing element 101d configured to direct a flow of aerosolized medication emitted (directly or indirectly) from the canister 102 towards the outlet 101b, as indicated by the arrow A. The housing 101 also comprises a locating portion 101e configured to receive the canister 102. The locating portion 101e acts to locate and retain the canister 102 in the correct position within the housing 101.

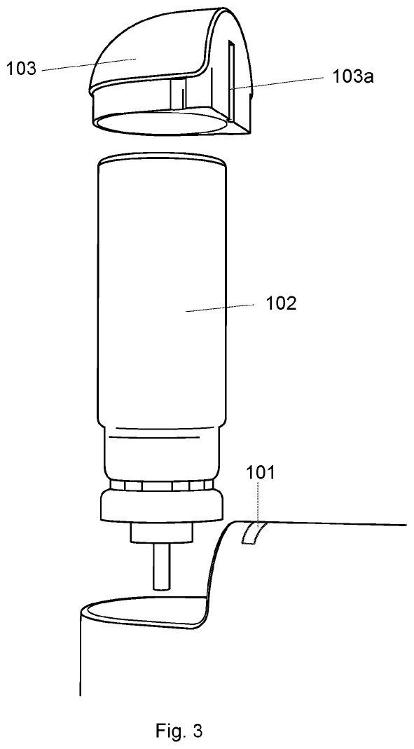

In the embodiment shown, the at least one flow directing element 101d is disposed adjacent or near to the canister 102. The at least one flow directing element 101d is shown in FIG. 1 as being a flat plate extending directly from an internal surface of the housing 101. In other embodiments, the at least one flow directing element 101d is a flat plate separated from an internal surface of the housing 101 by a stand or supporting section which does not act to direct a flow of aerosolized medication emitted from the canister 102 (see FIG. 2A). The stand or supporting section is shown as extending substantially perpendicularly from an internal surface of the housing 101 in FIG. 2A, although the skilled person will appreciate that the supporting section may extend in any direction. Instead of a plate, the at least one flow directing element 101d may be a wedge or other solid element with a flat surface. In alternative embodiments, the at least one flow directing element 101d is a curved plate, wedge or ridge as shown in FIGS. 2B and 2C. Similar to the embodiment shown in FIG. 2A, FIG. 2C shows the at least one flow directing element 101d separated from an internal surface of the housing 101 by a stand or supporting section. The at least one flow directing element 101d is disposed adjacent the canister 102.

The flow directing element(s) 101d may be angled with respect to an internal surface 101g of the housing 101. In the embodiment shown, the angle α of the flow directing element(s) relative to the internal surface 101g of the housing 101 is approximately 60°. In other embodiments, the angle of the or each flow directing elements 101d is between substantially 50° and substantially 70° relative to the internal surface 101g of the housing 101. Additionally or alternatively, in some embodiments an angle of one or more of the flow directing elements 101d is between 110° and 130° relative to the initial direction of flow of the aerosolized medication. Computational fluid dynamics may be utilised to determine an optimal angle for the/each flow directing element 101d, relative to the shape and/or dimensions of the housing 101 in which the flow directing element(s) 101d are implemented. As shown in the figures and in use, the internal surface 101g is a lower or bottom surface.

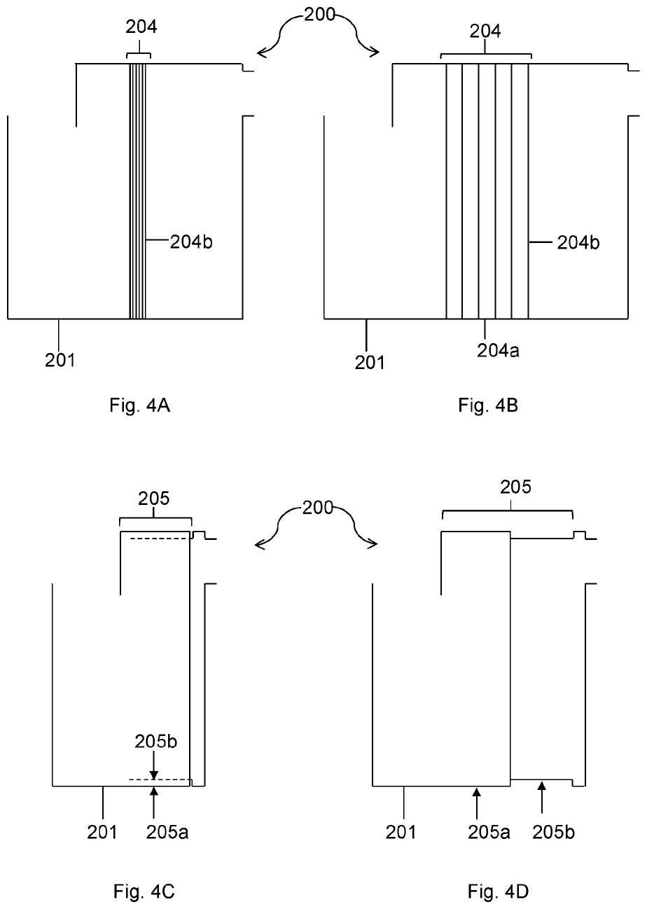

The locating portion 101e of the housing 101 locates and retains the canister 102 within the housing 101, but also enables the canister 102 to be removed or replaced, for example to replace an empty canister 102 with a full canister 102 as and when required. The locating portion 101e comprises an opening in an external surface of the housing 101 through which a canister 102 is received to locate the canister 102 within the housing 101. In the embodiment shown in FIG. 3, the inhaler 100 comprises a cap 103 configured to cover the canister 102 when it is located in the locating portion 101e. The cap 103 acts to seal the locating portion 101e when covering the canister 102. The cap 103 also acts as an actuator to actuate the canister 102 to emit aerosolized medication. The cap 103 may be depressed by a user to actuate the canister 102. When the cap is depressed by a user to actuate the canister 102, the cap 103 slides relative to the locating portion 101e of the housing 101. In the embodiment shown, when not depressed by a user to actuate the canister 102, the cap 103 is substantially flush with an external surface of the housing 101. In alternative embodiments, the cap 103 may not be flush with an external surface of the housing.

In the embodiment shown in FIG. 3, engagement features 103a on an external surface of the cap 103 are configured to interact with corresponding engagement features on an external surface of the housing 101 (not shown) to guide movement of the cap 103 during depression by a user. In the embodiment shown, the engagement features 103a are or comprise one or more female members or grooves on the external surface of the cap 103 configured to interact with a corresponding male member or tongue on an external surface of the housing 101, although the skilled person will appreciate other engagement features may be used instead to guide movement of the cap 103. In other embodiments, an internal surface of the locating portion 101e additionally or alternatively comprise engagement features configured to interact with corresponding engagement features on the external surface of the cap 103 e.g. the female member(s) may instead be provided on the housing and the male member(s) may be provided on the cap 103. In further alternative embodiments, no engagement features are present on the external surface of the cap 103 or the external surface of the housing 101 or the internal surface of the locating portion 101e. Instead, the cap 103 simply slides within the locating portion 101e when depressed by a user to actuate the canister 102 to emit aerosolized medication. In alternative embodiments, the inhaler 100 does not comprise a cap for the canister 102.

In the embodiment shown in FIG. 1, the housing 101 encloses a space 101a having a fixed volume. In some embodiments, the fixed volume is between substantially 100 ml and substantially 150 ml. In some embodiments, the fixed volume is substantially 120 ml.

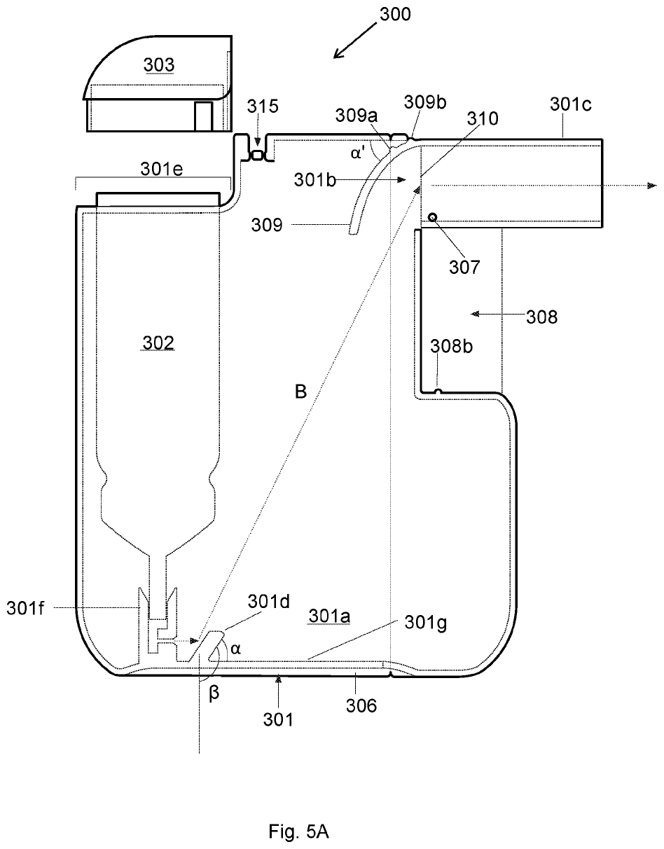

FIGS. 4A to 4D show embodiments of an inhaler 200 comprising a housing 201 having a volume that is alterable between a first volume and a second volume. The housing 201 comprises a mechanism configured to alter the volume of the housing 201 between the first volume and the second volume. The skilled person will appreciate that the housing 201 of the inhaler 200 may be substantially similar to and/or comprise some or all of the features of the housing 101. However, for simplicity, some features of the housing 101 depicted in FIG. 1 are omitted from FIGS. 4A to 4C in order to illustrate the mechanism 204.

FIGS. 4A and 4B show a housing 201 having a concertina mechanism 204 configured to alter the volume of the housing 201 between a first volume and a second volume. The concertina mechanism 204 comprises a plurality of portions 204a of a wall of the housing 201. Adjacent wall portions 204a are connected via a fold or pleat 204b. The concertina mechanism 204 is compressible or extendible by opening or closing the folds or pleats 204b to arrange the wall portions 204a in an extended or compressed state. The user may apply force to open or close the folds or pleats 204b. In the compressed state, as shown in FIG. 4A, the folds or pleats 204b are closed, the housing 201 having a first volume suitable for storage or transport. In the extended state, as shown in FIG. 4B, the folds or pleats 204b are open, the housing 201 having a second volume suitable for use to administer aerosolized medication to the user.

FIGS. 4C and 4D show a housing 201 having a telescopic mechanism 205 configured to alter the volume of the housing 201 between a first volume and a second volume. The telescopic mechanism 205 is configured to be moveable between an extended state and a compressed state. The telescopic mechanism 205 comprises a first connecting portion 205a configured to receive within it, in the compressed state, a second connecting portion 205b, as depicted by the dashed lines in FIG. 4C. The external dimensions of the second connecting portion 205b are therefore smaller than the internal dimensions of the first connecting portion 205a. The second connecting portion 205b is configured to be extended out of the first connecting portion 205a in order to move the telescopic mechanism 205 from the compressed state to the extended state. The user may apply force to move the second connecting portion 205b relative to the first connecting portion 205a. In the compressed state, as shown in FIG. 4C, the second connecting portion 205b is fully received within the first connecting portion 205a, the housing 201 having a first volume suitable for storage or transport. In the extended state, as shown in FIG. 4D, the second connecting portion 205b is fully extended out of the first connecting portion 205a, the housing 201 having a second volume suitable for use to administer aerosolized medication to the user. In some embodiments, the telescopic mechanism 205 comprises a stop preventing the second connecting portion 205b from being fully removed from the first connecting portion 205a. In some embodiments, the telescopic mechanism 205 comprises a seal to prevent loss of air or aerosolized medication through the telescopic mechanism 205.

In alternative embodiments, a mechanism configured to alter a volume of the housing 201 between a first volume and a second volume comprises two or more housing portions which are rotatable with respect to one another, relative rotation between the housing portions causing a volume of the housing 201 to alter (for example, via one or more protrusions on one or more of the housing portions configured to interact with one or more helical grooves on one or more of the other housing portions to drive the housing portions together or apart from one another). The skilled person will appreciate that other mechanisms for altering a volume of the housing 201 may be implemented.

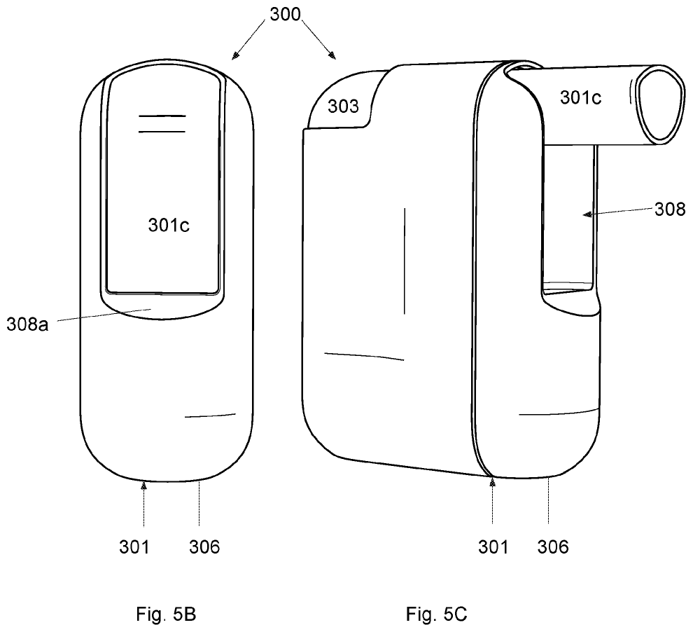

FIGS. 5A to 5C show an inhaler 300 in accordance with an embodiment of the invention. The inhaler 300 comprises a housing 301 and an aerosolized medication canister 302. A cap 303 is provided to cover the canister 302, although in alternative embodiments a cap is not present. The skilled person will appreciate that the housing 301 of the inhaler 300 may be substantially similar to and/or comprise some or all of the features of the housing 101 of the inhaler 100 and/or the housing 201 of the inhaler 200 and/or the features described with respect to the inhaler 300 may be used in the housing 101/inhaler 100 and/or the housing 201/inhaler 200.

The housing 301 comprises a main body 306. The main body 306 comprises an outlet 301b. The outlet 301b comprises a mouthpiece 301c. In the embodiment shown, the mouthpiece 301c is substantially tubular, having a cross-section approximated by a square with rounded edges. In alternative embodiments, a cross-section of the mouthpiece 301c is a different shape, for example a square shape, a rectangular shape, a circular shape, an oval shape or an ellipse shape. In some embodiments, the cross-section of the mouthpiece 301c is selected to substantially match a shape of an outer surface of the main body 306 of the housing 301. This is discussed in more detail below.

The mouthpiece 301c is moveably secured to the main body 306. The mouthpiece 301c is moveable between a first position in which the outlet 301b is open, and a second position in which the outlet 301b is closed. In the embodiment shown, the mouthpiece 301c is secured to the main body 306 using a pivot or hinge 307. The mouthpiece 301c is configured to rotate about the pivot or hinge 307 between the first position and the second position. In the embodiment shown, the main body 306 of the housing 301 comprises a recess 308 in an outer surface which is configured to receive the mouthpiece 301c when the mouthpiece 301c is in the second position. The mouthpiece 301c is therefore substantially flush with an outer surface of the main body 306 of the housing 301 when the mouthpiece 301c is in the second position. The ‘flushness’ of the mouthpiece 301c with an outer surface of the main body 306 of the housing 301 is maximised when a cross-section of the mouthpiece 301c provides an outer surface of the mouthpiece 301c that is substantially similar to a shape of an outer surface of the main body 306 of the housing 301 at or near the mouthpiece 301c. This is illustrated more clearly in FIG. 5B. FIG. 5B also illustrates a notch or chamfer 308a which is disposed on an outer surface of the main body 306 of the housing 301 adjacent the recess 308. The notch 308a enables a user to access an end of the mouthpiece 301c in order to apply force to move the mouthpiece 301c from the second position to the first position, as shown in FIGS. 5A and 5C.

Returning to FIG. 5A, the recess 308 comprises a stop or retaining element 308b. The stop 308b is configured to interact with an open end of the mouthpiece 301c. In order to place the mouthpiece 301c in the second position to close the outlet 301b, the open end of the mouthpiece 301c must pass over the stop 308b. When an outer surface of the mouthpiece 301c meets the stop 308b whilst placing the mouthpiece in the second position, continued application of force to the mouthpiece 301c will cause elastic deformation of either the stop 308b or the mouthpiece 301c, enabling the mouthpiece 301c to flex and pass over the stop 308b. The size and dimensions of the stop 308 (which in part determine the stiffness of the stop 308b, together with the inherent material properties of the stop 308b) determine the force needed to be applied to move the mouthpiece 301c over the stop 308b. Once the mouthpiece 301c has passed over the stop 308b, the mouthpiece 301c is securely retained within the recess 308 in the second position, closing the outlet 301b. To return the mouthpiece 301c to the first position and open the outlet 301b, a user applies force to the mouthpiece 301c by accessing the open end of the mouthpiece 301c via the notch 308a to move the mouthpiece 301c over the stop 308b in the opposite direction.

In alternative embodiments (not shown), the mouthpiece 301c is configured to slide linearly in and out of the outlet 301b in order to open and close the outlet 301b. In some embodiments, guide rails may be provided on an internal surface of the housing 301 to guide movement of the mouthpiece 301c through the outlet 301b. One or more protrusions extending from the external surface of the mouthpiece 301c may engage with the guide rails to guide movement of the mouthpiece 301c through the outlet 301b in a linear manner.

In the embodiment shown in FIG. 5A, the mouthpiece 301c comprises at least one flow directing element 309. The at least one flow directing element 309 is configured to direct a flow of aerosolized medication towards the outlet 301b, when the mouthpiece 301c is in the first position (i.e., the outlet 301b is open). In the embodiment shown, the at least one flow directing element 309 comprises a curved plate. In alternative embodiments, the at least one flow directing element 309 comprises a different shape such as a flat plate, similar to that described above for the at least one flow directing element 101d disposed within the housing 101 (see FIG. 1). In the embodiment shown, the at least one flow directing element 309 is located in the space 301a enclosed by the housing 301 (when the mouthpiece 301c is in the first position). The at least one flow directing element 309 is configured to interact with the at least one flow directing element 301d to form a primary or direct flow path B (shown in dotted lines) for aerosolized medication emitted from the canister 102 through the space 301a enclosed by the housing 301. In embodiments in which the mouthpiece 301c is not moveable between a first position and a second position (such as the mouthpiece 101c of the inhaler 100), the skilled person will appreciate that the mouthpiece 301c may still comprise at least one flow directing element 309. For example, the at least one flow directing element 309 could extend from the mouthpiece 301c into the space 301a enclosed by the housing 301 and provide substantially the same effect. Alternatively, the at least one flow directing element 309 may not be a part of the mouthpiece 301c, but may extend from an internal surface of the housing 301 adjacent the mouthpiece 301c to provide substantially the same effect. In some embodiments, the housing 301 may not comprise the at least one flow directing element 301d disposed adjacent to the canister 302.

In some embodiments, the spatial relationship between the at least one flow directing element 301d and the at least one flow directing element 309 determines the structural arrangement of the flow directing elements 301d, 309. For example, in some embodiments, the angle of the flow directing elements 301d, 309 relative to an internal surface of the housing 301 is substantially similar or identical such that the flow directing elements 301d, 309 are substantially parallel to one another (as depicted in FIG. 5A). Arranging the flow directing elements 301d, 309 substantially parallel to one another may provide a substantially linear primary flow path for aerosolized medication through the space 301a enclosed by the housing 301. The angle may depend on a distance between the flow directing elements 301d, 309. In the embodiment shown, the angle α of the at least one flow directing element 301d relative to an internal surface 301g of the housing is approximately 60°. The angle 'α of the at least one flow directing element 309 relative to an internal surface of the housing 301 is also approximately 60°. In other embodiments, the angle of each of the at least one flow directing elements 301d, 309 is between substantially 50° and substantially 70° relative to an internal surface of the housing 301. Additionally or alternatively, in some embodiments an angle of one or more of the flow directing elements 301d is between 110° and 130° relative to an initial direction of flow of the aerosolized medication. A greater distance between the flow directing elements 301d, 309 would require a shallower angle of the flow directing elements 301d, 309 (for example, closer to parallel to an internal surface of the housing 301 than to perpendicular to an internal surface of the housing 301), and vice versa. In the embodiment shown, a lateral distance across a width of the housing 301 between the flow directing elements 301d, 309 is substantially 20 mm. In other embodiments, a lateral distance across a width of the housing 301 between the flow directing elements is between 15 mm and 40 mm. In some embodiments, an angle of the flow directing element 301d relative to an internal surface of the housing 301 may be different from an angle of the flow directing element 309 relative to an internal surface of the housing 301. Computational fluid dynamics may be utilised to determine an optimal angle for each of the at least one flow directing element 301d and the at least one flow directing element 309, relative to the shape and/or dimensions of the housing 301 in which the flow directing elements 301d, 309 are implemented.

An angle of the at least one flow directing element 301d relative to an internal surface of the housing 301 also depends on a distance (for example, a lateral distance across a width of the housing 301) between the locating portion 301e, the canister 302 or an actuator nozzle 301f and the at least one flow directing element 301d (or such a distance as a proportion of the total lateral distance between the flow directing elements 301d, 309). The same consideration applies to the at least one flow directing element 309.

In the embodiment shown, a lateral distance across a width of the housing 301 between the at least one flow directing element 301d and the actuator nozzle 301f is approximately 5 mm. In other embodiments, a lateral distance across a width of the housing 301 between the at least one flow directing element 301 and the actuator nozzle 301f, the locating portion 301e or the canister 302 may be between substantially 3 mm and substantially 25 mm, and in particular is between substantially 5 mm and substantially 15 mm. A lateral distance across a width of the housing 301 between the at least one flow directing element 309 and the outlet 301b is approximately 5 mm in the embodiment shown. In other embodiments, a lateral distance across a width of the housing 301 between the at least one flow directing element 309 and the outlet 301b is between substantially 3 mm and substantially 25 mm, and in particular is between substantially 5 mm and substantially 15 mm.

In some embodiments, the actuator nozzle 301f aids location and retention of the canister 302 in a locating portion 301e of the housing 301. The actuator nozzle 301f retains an elongate delivery tube or valve extending from the canister 102, thereby further reducing movement of the canister 102 once located in the locating portion 301e of the housing 301. In some embodiments, the actuator nozzle 301f extends from an internal surface of the housing 301. The actuator nozzle 301f is configured to atomise liquid medication emitted from the canister 102 to produce aerosolized medication. In some embodiments, the medication is not aerosolized unless it passes through the actuator nozzle 301f (that is, the actuator nozzle 301f is what actually produces aerosolized medication from medication held within the canister 102). In some embodiments, the actuator nozzle 301f is a standard actuator nozzle typically used to interact with a standard aerosolized medication canister 102 in order to produce aerosolized medication.

In some embodiments, the flow directing elements 301d, 309 extend substantially between opposite internal surfaces of the housing 301 (for example, across a thickness or width of the space 301a enclosed by the housing 301). In other embodiments, the flow directing elements 301d, 309 extend only partially across a thickness or width of the space 301a enclosed by the housing 301. Similar to the embodiment of FIGS. 1 and 2, each of the at least one flow directing elements 301d may a flat plate extending directly from the internal surface 301g of the housing 301. In other embodiments, the at least one flow directing element 301d is a flat plate separated from an internal surface of the housing 301 by a stand or supporting section which does not act to direct a flow of aerosolized medication emitted from the canister 302 (similar to FIG. 2A). Instead of a plate, the at least one flow directing element 301d may be a wedge or other solid element with a flat surface. In alternative embodiments, the at least one flow directing element 301d is a curved plate, wedge or ridge. The at least one flow directing element 301d may be separated from the internal surface 301g of the housing 301 by a stand or supporting section.

In the embodiment shown, the at least one flow directing element 309 is also configured, shaped or arranged to form part of a sealing structure that closes the outlet 301b when the mouthpiece 301c is in the second position. When the mouthpiece 301c is in the second position, an end (for example, an end furthest away from the tubular structure of the mouthpiece 301c) of the at least one flow directing element 309 is positioned to contact the main body 306 of the housing 301, adjacent the outlet 301b, to ensure that the housing 301 is substantially sealed. In some embodiments, a pair of stops or retaining elements 309a, 309b are provided on the at least one flow directing element 309 and configured to interact with the outlet 301b, adjacent the main body 306 of the housing 301, to aid retention of the mouthpiece 301c in the second position. A lip of the outlet 301b is configured to be retained between the stops 309a, 309b. The stops 309a, 309b function similarly as described above in respect of stop 308b. In some embodiments, the at least one flow directing element 309 does not comprise stops or retaining elements to maintain the mouthpiece 301c in the second position. Therefore, the at least one flow directing element 309 is configured to perform different roles, depending on whether the mouthpiece 301c is in the first position or the second position.

In the embodiment shown in FIG. 5A, the mouthpiece 301c comprises a membrane 310. The membrane 310 comprises a valve. The valve of the membrane 310 is configured to provide low resistance to flow when the user is inhaling aerosolized medication through the mouthpiece 301c. In the embodiment shown, the membrane 310 extends substantially across the full cross-sectional area of the mouthpiece 301c. As such, the membrane 310 has a shape corresponding to an internal shape of the mouthpiece 301c. The presence of the membrane 310 means that flow of aerosolized medication and air must pass through the membrane 310 and the valve, and is unlikely to bypass or flow around the membrane 310. The skilled person will appreciate that a similar membrane could be implemented in the inhalers 100, 200 described above. In alternative embodiments, the mouthpiece 301c does not comprise a membrane 310.

The inhaler 300 further comprises a whistle structure 315 in the embodiment shown in FIG. 5A. The whistle structure 315 is disposed in a wall of the housing 301. In the embodiment shown, the whistle structure 315 comprises two closely spaced, elongate apertures through the wall of the housing 301. The whistle structure 315 is configured to produce a high-pitched noise if, during use, a user inhales too quickly. Rapid inhalation may cause a flow rate of air and/or aerosolized medication through the space 301a enclosed by the housing 301 to exceed a threshold. This can reduce the effective dose of medication. Using feedback from the whistle structure 315, the user can modulate their inhalation to ensure optimal use of the inhaler 300 to maximise an effective dose of aerosolized medication.

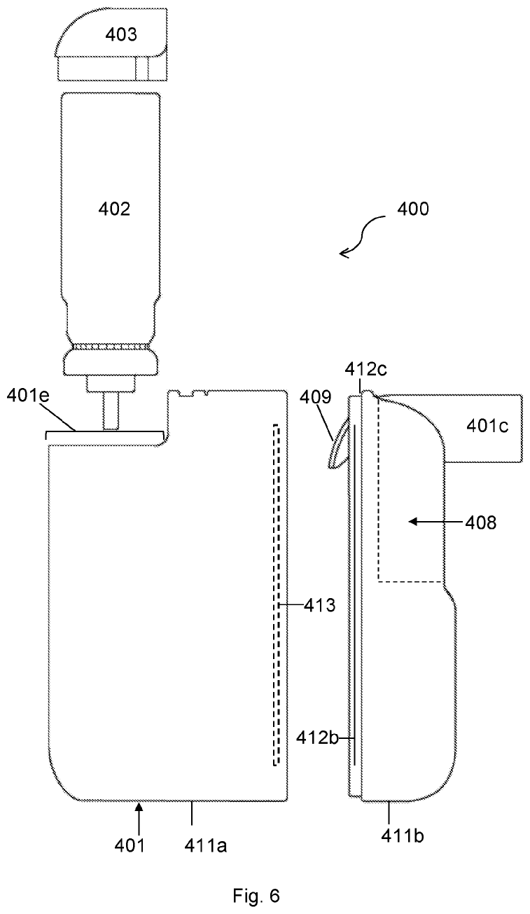

FIG. 6 shows an inhaler 400 in accordance with another embodiment of the invention. The inhaler 400 is substantially similar to the inhaler 300 described above with respect to FIGS. 5A to 5C, and like reference numerals have been used. However, the skilled person will appreciate that the following could equally apply to the inhalers 100, 200 described above and/or features of those embodiments may be utilised here. The main body 406 of the housing 401 of the inhaler 400 comprises a plurality of housing portions 411a, 411b. In the embodiment shown, the housing 401 comprises two housing portions, but the skilled person will appreciate that a greater number of housing portions could be used. A first housing portion 411a is configured to releasably attach to a second housing portion 411b. In the embodiment shown, a total width of the housing (from the left-most edge to the right-most edge in FIG. 6) is substantially 75 mm. A width of the first housing portion 411a is substantially 50 mm, and a width of the second housing portion is substantially 25 mm. In the embodiment shown, an open end of the second housing portion 411b comprises a region 412 having external dimensions less than the internal dimensions of an open end of the first housing portion 411a. This enables the region 412 to be received within the first housing portion 411a. The difference between the internal dimensions of the open end of the first housing portion 411a and the external dimensions of the region 412 of the second housing portion 411a is small enough that an internal surface of the first housing portion 411a is in contact with an outer surface of the region 412 when the region 412 is received within the first housing portion 411a, but large enough to allow sliding movement between the contacting surfaces (for example, without the application of significant external force such as in a push-fit or friction-fit). The region 412 also has reduced external dimensions relative to an outer surface of the second housing portion 411b. This creates a shoulder structure 412c on the second housing portion 411b. The external dimensions of the second housing portion 411b where the region 412 forms the shoulder structure 412c are substantially identical to the external dimensions of the first housing portion 411a. Therefore, the region 412 can only be received in the first housing portion 411a up until the first housing portion 411a comes into contact with the shoulder structure 412c. The shoulder structure 412c prevents the second housing portion 411b from moving any further inside the first housing portion.

On an external surface of the region 412, an engagement feature 412b is disposed. In the embodiment shown, the engagement feature 412b is a male member such as a flange or tongue extending or protruding from the external surface of the region 412. As the region 412 is moved inside the first housing portion 411a, the flange 412b is configured to elastically deform (for example, compress or deflect). However, a corresponding (female) engagement feature 413 is disposed on an internal surface of the first housing portion 411a, with which the engagement feature 412b is configured to interact. In the embodiment shown, the engagement feature 413 is a groove configured to receive the flange 412b. When the region 412 has moved a sufficient distance inside the first housing portion, the compressed or deflected flange 412b meets the groove 413. On meeting the groove 413, the compressed or deflected flange 412b returns to its original shape and structure and is retained within the groove 413. The corresponding engagement features 412b, 413 prevent the housing portions 411a, 411b from becoming inadvertently separated once attached to one another. The user may need to apply force (for example, by squeezing one or both of the housing portions 411a, 411b) to elastically deform one or both of the engagement features 412b, 413 in order to detach the second housing portion 411b from the first housing portion 411a (for example, to remove the flange 412b from the groove 413). In some embodiments, the attachment between the first housing portion 411a and the second housing portion 411b is sufficient to substantially seal the housing to prevent loss of air or aerosolized medication through the join between the housing portions 411a, 411b (and also to prevent ingress of external, foreign matter into the housing 401). Alternatively, in some embodiments, a seal (for example, an o-ring seal) is disposed on one or both of an external surface of the region 412 and an internal surface of the first housing portion 411b in order to prevent loss of air or aerosolized medication through the join between the housing portions 411a, 411b.

The skilled person will appreciate other techniques or features for removably attaching two or more housing portions together could be implemented. In some embodiments, the region 412 does not comprise an engagement feature 412b. In some embodiments, the region 412 is instead received within the first housing portion 411a using a push-fit or friction-fit to attach the two housing portions 411a, 411b. In alternative embodiments, resilient elastic elements (for example, sprung clips) on one or both of the housing portions 411a, 411b are arranged and configured to elastically deform to move over a flange disposed on an internal surface of the other of the one or both housing portions 411a, 411b in order to attach the housing portions 411a, 411b. In some embodiments, a seal may be located on one or both of the housing portions 411a, 411b to prevent loss of air or aerosolized medication through the join between the housing portions 411a, 411b (and to prevent ingress of external, foreign matter into the housing 401). In some embodiments instead the male/female features being provided on the housing portions 411b/411a respectively, the male/female features may be provided on the housing portions 411a/411b respectively.

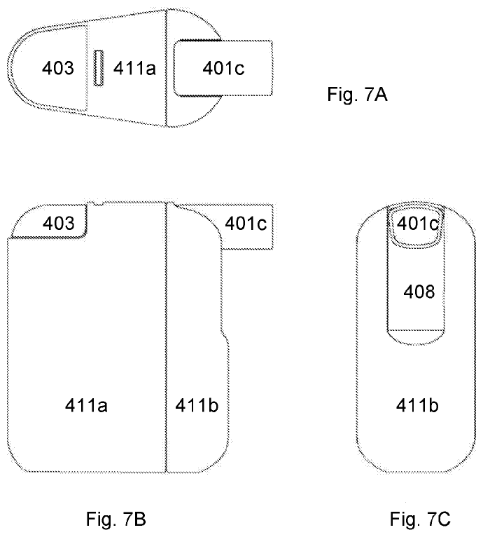

FIGS. 7A to 7C show various views of the inhaler 400, indicating dimensions of the inhaler 400. The skilled person will appreciate that those dimensions are illustrative of a concept and are not limiting in any way. The skilled person will also appreciate that the dimensions discussed are equally applicable to the inhalers 100, 200, 300 discussed above. For the inhaler 200, the external dimensions discussed may be representative of external dimensions of the inhaler 200 when in an extended state.

FIG. 7A indicates the mouthpiece 401c has a width of approximately 19 mm. FIGS. 7B and 7C together indicate that the overall dimensions of the inhaler 400 are approximately 92 mm×75 mm×40 mm (height×width×depth). In the embodiment shown, an overall shape of the inhaler 400 is substantially teardrop shaped (as shown in the plan view in FIG. 7A). The teardrop shape, in the embodiment shown, comprises a tapering from one end to another, and rounded or chamfered edges. A teardrop shape extends in depth or thickness from the location of a canister 402 within the housing 401 to a location of an outlet 401b in the housing 401. A teardrop shape may improve flow of aerosolized medication through the space 401a enclosed by the housing 401. Considering the tapering of the depth of the inhaler from the front (for example, where the mouthpiece 401c is disposed) to the back, and the rounded or chamfered external edges of the inhaler 400, the volume of space 401a enclosed by the housing 401 of the inhaler 400 is approximately 120 ml. In some embodiments, external dimensions of the inhaler 400 are between 80 mm×60 mm×30 mm and 100 mm×90 mm×50 mm. In some embodiments, a volume of space 401 enclosed by the housing 401 of the inhaler 400 is between 80 ml and 200 ml, and in other embodiments is between 100 ml and 150 ml. Such dimensions and volumes are an optimal compromise for an inhaler that is portable and easy to carry around (for example, able to fit in a typical garment pocket) whilst still providing a large enough volume to increase an effective dose of aerosolized medication similarly to a typical separate spacer device.

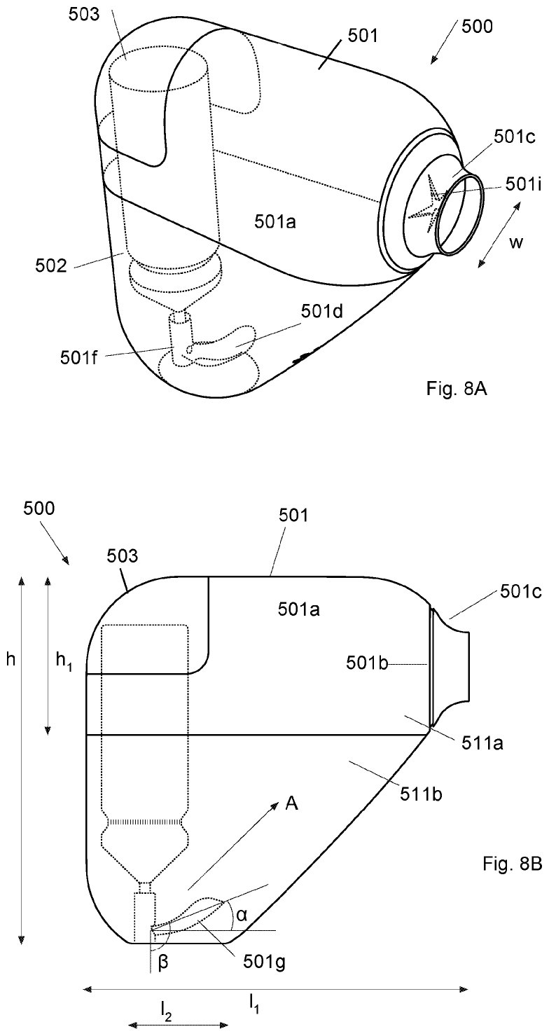

FIGS. 8A and 8B show an alternative embodiment of an inhaler 500 in accordance with the invention. The inhaler 500 comprises a housing 501 enclosing a space 501a. The inhaler 500 also comprises an aerosolized medication canister 502. The housing 501 comprises an outlet 501b. The outlet 501b comprises a mouthpiece 501c. The housing 501 also comprises at least one flow directing element 501d configured to direct a flow of aerosolized medication emitted (directly or indirectly) from the canister 502 towards the outlet 501b, as indicated by the arrow A. The housing 501 may also comprises a locating portion similar to locating portion 101e of FIG. 1.

The inhaler housing 501 is substantially triangular in lateral (side) cross section, i.e. through the height and length of the inhaler housing 501. This provides the housing with a narrow triangular prism configuration. As shown in FIG. 8B, the upper surface of the housing 501 has a greater length than the lower surface of the housing 501. The housing tapers from the lower surface towards the mouthpiece 501c at an angle of substantially 44°. Alternatively the housing may taper from the lower surface towards the mouthpiece 501c at an angle of substantially 44-46°, 42-48 or 40-50°.

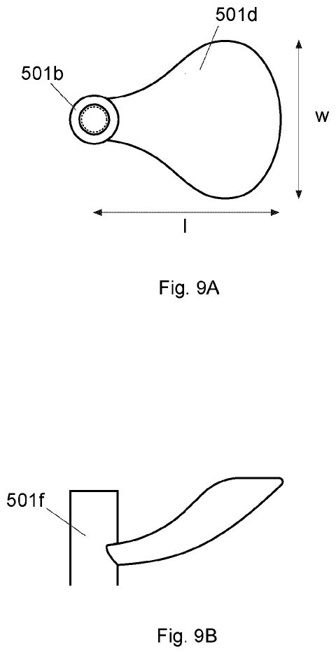

FIGS. 9A and 9B show the flow directing element 501d. In the embodiment shown, the at least one flow directing element 501d is disposed adjacent or near to the canister 502. The at least one flow directing element 501d is shown in FIGS. 8A and 9B as being concave, similar to FIG. 2C. The at least one flow directing element 501d extends from a supporting section 501f provided on an interior surface of the housing 501 and into which the canister 502 is inserted. In the embodiment shown the supporting section 501f is cylindrical, which is convenient for receiving the canister 502, but other shapes and configurations could be used. The supporting section 501f may be configured with an actuator nozzle as shown in FIG. 5A, for example, and as discussed above.

In some embodiments, the at least one flow directing element 501d extends substantially between opposite internal surfaces of the housing 501 (for example, across a thickness or width of the space 501a enclosed by the housing 501). In other embodiments, the at least one flow directing element 501d extends only partially across a thickness or width of the space 501a enclosed by the housing 501. The at least one flow directing element 501d may have a width w of substantially 14-20 mm, or 15-19 mm, or 16-18 mm, and preferably 17 mm across the widest part. The at least one flow directing element 5 may have a length 1 of substantially 15-25 mm, or 16- or 17-23 mm, or 18-22 mm, or 19-21 mm and preferably 20 mm from the centre of the canister support 501f to the opposite end. The concave shape of at least one flow directing element 501d is similar to a ski slope and is configured to regulate the flow of aerosol medication from the canister 502. In an alternative embodiment (not shown), the at least one flow directing element 501d may have a twisted or spiral configuration, or comprise a plurality of differently curved surfaces and/or be asymmetric. These configurations provide for additional mixing and turbulence within the housing 501. The at least one flow directing element 501d may instead be of any of the configurations shown or described above in relation to earlier embodiments.

The flow directing element(s) 501d may be angled with respect to an internal surface 501g of the housing 501. In the embodiment shown, the angle α of the flow directing element(s) relative to the internal surface 101g of the housing 101 is approximately 25°. In other embodiments, the angle of the or each flow directing elements 501d is between substantially 15° and substantially 40° relative to the internal surface 501g of the housing 501, or between substantially 15° and substantially 35° relative to the internal surface 501g of the housing 501, or substantially 20° and substantially 30° relative to the internal surface 501g of the housing 501. In the embodiment shown, the angle of the flow directing element(s) relative to an initial direction of flow of aerosolized medication is approximately 115°. In other embodiments, the angle is between substantially 85° and substantially 135° relative to an initial direction of flow of aerosolized medication, or substantially 95° and substantially 130° relative to an initial direction of flow of aerosolized medication, or substantially 105° and substantially 125° relative to an initial direction of flow of aerosolized medication, or substantially 110° and substantially 120° relative to an initial direction of flow of aerosolized medication. As above, computational fluid dynamics may be utilised to determine an optimal angle for the/each flow directing element 501d, relative to the shape and/or dimensions of the housing 501 in which the flow directing element(s) 501d are implemented.

As in earlier embodiments, at least one flow directing element may be provided at or in the vicinity of the outlet 501b.

Similar to earlier embodiments, an opening in an external surface of the housing 501 is provided through which a canister 502 is received to locate the canister 502 within the housing 501. In the embodiment shown in FIGS. 8A and 8B, the inhaler 500 comprises a cap 503 configured to cover the canister 502 when it is located in the housing 501. The cap 503 and its operation is similar to that described above.

The inhaler 500 may comprise any feature described above in connection with earlier embodiments.

In the embodiment shown in FIGS. 8A and 8B, the housing 501 encloses a space 501a having a fixed volume. In an embodiment, the volume capacity of the housing 501 is substantially 100-140 ml, or 110-130 ml, or 120 ml.

In an embodiment, the housing 501 has a height of substantially 85-105 mm, 88-97, or 92-95 or 93.5 mm. In an embodiment, the housing 501 has a major length l₁ (along the upper surface) of substantially 90-105 mm, or 93-102 mm, or 95-100, or 97-98 mm, 97.5 mm. In an embodiment, the housing 501 has a minor length l₂ (along the lower surface) of substantially 22-26 mm, or mm. In an embodiment, the mouthpiece 501c has a width w of substantially 15-20 mm, or 16-19 mm, or 17-18 mm, or 17.5 mm.

The mouthpiece 501c is substantially conical, having a substantially circular cross-section. In alternative embodiments, a cross-section of the mouthpiece 501c is a different shape, for example a square shape, a rectangular shape, an oval shape or an ellipse shape. In some embodiments, the cross-section of the mouthpiece 501c is selected to substantially match a shape of an outer surface of the main body 506 of the housing 501. Similar to the description above in relation to FIG. 5A, the mouthpiece 501c may comprise a filtering mechanism or membrane. The filtering mechanism comprises a valve 501i. The valve 501i of the membrane is configured to provide low resistance to flow when the user is inhaling aerosolized medication through the mouthpiece 501c. The filtering mechanism may extend substantially across the full cross-sectional area of the mouthpiece 501c. As such, the filtering mechanism 510 has a shape corresponding to an internal shape of the mouthpiece 501c. The presence of the filtering mechanism means that large particles of aerosolized medication do not pass through the filtering mechanism and the valve 501i, and is unlikely to bypass or flow around the filtering mechanism. The valve 501i may be an inspiratory valve.

The mouthpiece 501c may be attachable to and detachable from the housing 501, e.g. for cleaning. Complementary screwthreads may be provided on the interior of the mouthpiece and on the exterior of the outlet 501b. Other known attachment means may alternatively be provided e.g. snap or press fit.

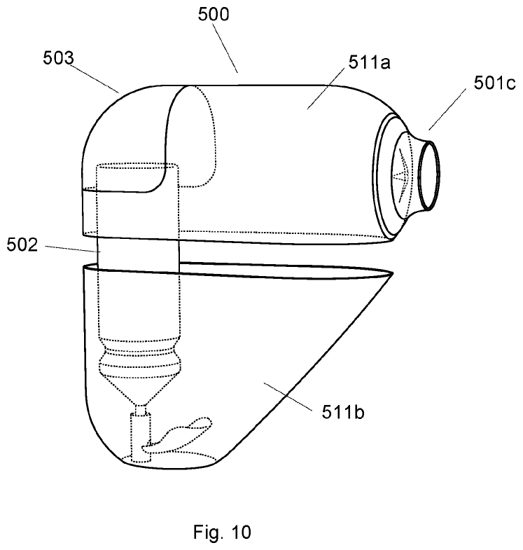

The housing 501 of the inhaler 500 of FIGS. 8A and 8B comprises a plurality of housing portions 511a, 511b, also shown in FIG. 10. The skilled person will appreciate that a greater number of housing portions could alternatively be used. A first housing portion 511a is configured to releasably attach to a second housing portion 511b. An engagement feature such as a male/female connector could be used. The male/female engagement feature may be provided on one of the housing portions and be receivable or lockable in the other female/male connector on the other housing portion. Alternatively, a push-fit or friction-fit could be used to attach the two housing portions 511a, 511b. In alternative embodiments, resilient elastic elements (for example, sprung clips) on one or both of the housing portions 511a, 511b are arranged and configured to elastically deform to move over a flange disposed on an internal surface of the other of the one or both housing portions 511a, 511b in order to attach the housing portions 511a, 511b. In some embodiments, a seal may be located on one or both of the housing portions 511a, 511b to prevent loss of air or aerosolized medication through the join between the housing portions 511a, 511b (and to prevent ingress of external, foreign matter into the housing 501). In some embodiments one or more male/female features may be provided on the housing portions 511a/511b respectively, or on the housing portions 511b/511a respectively. Alternatively, the attachment of housing portions 511a, 511b may be as described above in relation to FIG. 6. The first housing portion 511a may have a height h₁ of substantially 35-46 mm. In this embodiment, the cap 503 clips or locks into the upper part of the body 511a e.g. in a manner similar to that described in earlier embodiments.

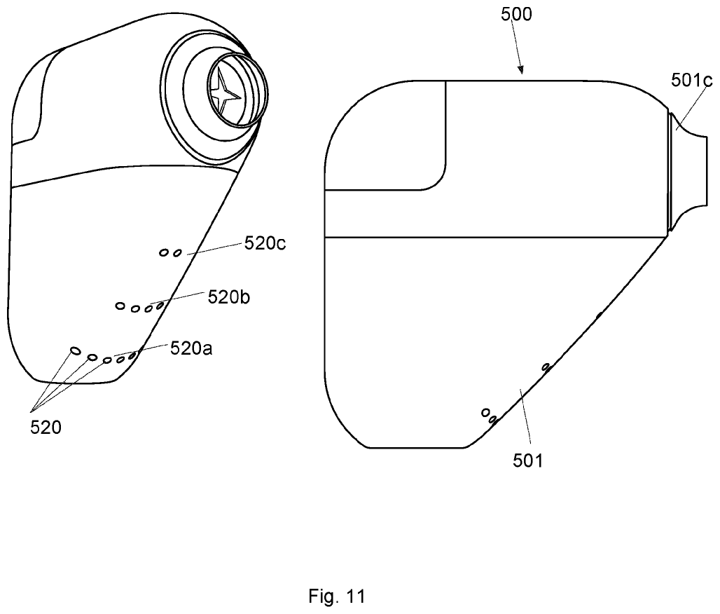

FIG. 11 illustrates an embodiment where the housing 501 is provided with a plurality of expiratory vents 520. In the embodiment shown, the vents 520 are positioned between the mouthpiece 501c and the lower surface of the device 500, and would face the user, in use. Positioning the vents 520 on this front surface is advantageous since they vents 520 have little or no effect on the aerosol flow. It also fulfils ergonomic requirements. The vents 520 fulfil the function of a spacer, meaning the user can breathe in and out through the device 500 during the process of taking the medication.

In the embodiment shown, three rows 520a, 520b, 520c each comprising a plurality of vents 520 are provided. Here, a total of 12 vents 520 are provided with six in the first row 520a, 4 in the second row 520b and two in the third row 520c. In the embodiment shown the separation between adjacent rows is substantially 9 mm, but could alternatively be 4 mm-14 mm, or 6 mm-12 mm or 8 mm-10 mm. The vents 520 are arranged symmetrically across an imaginary central line running along the front surface of the housing 501. In the embodiment shown, the diameter of each vent is substantially 1.5-3 mm, or 1.75-2.25 mm, or 1.9-2.1 mm, or 2 mm. The spacing between adjacent vents/holes 520 may be substantially the same or larger e.g. up to substantially 5 mm. The provision of a plurality of small vents 520 provides sufficient open area enabling the user to breathe whilst avoiding loss of medication from within the housing 501.

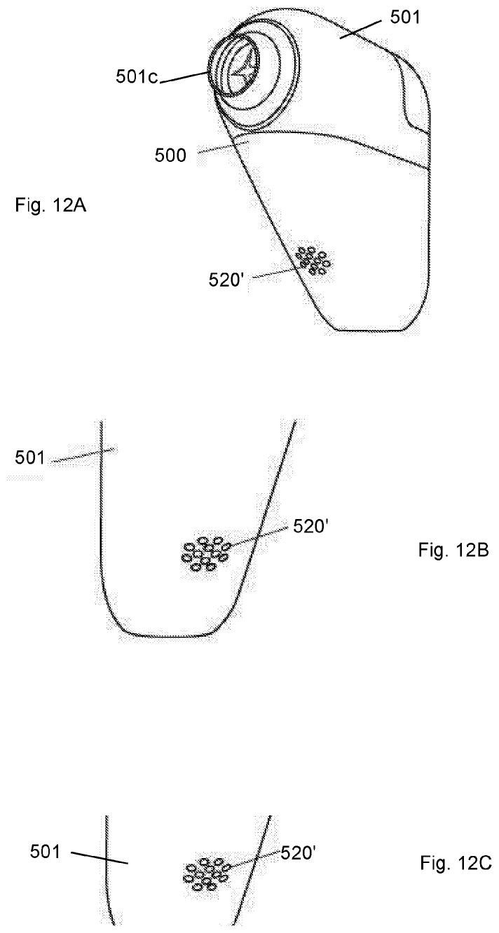

FIGS. 12A, 12B and 12C illustrates an alternative embodiment of a housing 501 is provided with a plurality of expiratory vents 520. In the embodiment shown, the vents 520 are again positioned between the mouthpiece 501c and the lower surface of the device 500, and would face the user, in use. In the embodiment shown, a plurality of vents 520 are provided in a regular, circular pattern. Here, a total of 12 vents 520 are provided with four in the centre and eight surrounding them, but it will be appreciated that fewer or more vents could be used and in a single or more (substantially concentric) rings. In the embodiment shown the diameter of each vent is again substantially 1.5-3 mm, or 1.75-2.25 mm, or 1.9-2.1 mm, or 2 mm. The spacing between adjacent vents/holes 520 may be substantially the same or larger e.g. up to substantially 5 mm. The vents are located approximately 16-25 mm from the lower surface of the device 500, although other locations could be used.

The rounded or chamfered external edges of the inhaler 300, 400, 500 also provide comfort for the user when held in the hand, and reduce a likelihood of the inhaler 300, 400, 500 ‘catching’ on a pocket or being uncomfortable to carry in a pocket. The substantially cuboidal shape of the inhaler 400 enables the inhaler to be easily stored in a pocket or bag relative to, for example, an inhaler of a similar volume but having a substantially cubic shape. The substantially triangular shape of the inhaler 500 was chosen to optimise flow of aerosol medicament inside as well as meet ergonomic requirements.

In some embodiments, the inhalers 100, 200, 300, 400 or one or more components of the inhalers are manufactured using an injection moulding process. In alternative embodiments, a different manufacturing process is utilised such as rotational moulding or blow moulding. In some embodiments, the inhalers 100, 200, 300, 400 are or comprise a polymer material or plastic (for example, polypropylene or polycarbonate). The typical properties of polymer materials or plastic make them well suited to inhalers, owing to their low density, high strength to weight ratio and toughness. In some embodiments, the polymer material comprises an antistatic material to prevent aerosolized medication from being electrostatically attracted to an internal surface of the housing 101, 201, 301, 401. In some embodiments, the polymer material is or comprises an antistatic material. Alternatively, the polymer material is coated with an antistatic coating to provide the same effect. In the same vein, polymer materials may provide antimicrobial and/or antibacterial effects. In some embodiments, the polymer material of the housing 101, 201, 301, 401 comprises an antibacterial or antimicrobial material or coating layer. This antimicrobial and/or antibacterial material or layer provides an additional layer of protection to the sealed housing 101, 201, 301, 401 designed to prevent ingress of foreign matter (such as dust, impurities etc.) into the housing 101, 201, 301, 401.

From reading the present disclosure, other variations and modifications will be apparent to the skilled person. Such variations and modifications may involve equivalent and other features which are already known in the art of inhalers, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination. The applicant hereby gives notice that new claims may be formulated to such features and/or combinations of such features during the prosecution of the present application or of any further application derived therefrom. Features of the devices and systems described may be incorporated into/used in corresponding methods.

For the sake of completeness, it is also stated that the term “comprising” does not exclude other elements or steps, the term “a” or “an” does not exclude a plurality, a single processor or other unit may fulfil the functions of several means recited in the claims and any reference signs in the claims shall not be construed as limiting the scope of the claims.

1-23. (canceled)

24. An inhaler housing comprising:
a locating portion configured to receive an aerosolized medication canister;
an outlet comprising a mouthpiece; and
at least one flow directing element disposed within the housing;
wherein the at least one flow directing element is configured to direct a flow of aerosolized medication toward the outlet; and
wherein a space enclosed by the housing is configured to reduce a velocity of said aerosolized medication prior to said aerosolized medication reaching the outlet for inhalation by a user.

25. The inhaler housing of claim 24, wherein the housing is configured to enclose a fixed volume of space.

26. The inhaler housing of claim 24, wherein the housing is configured to enclose an alterable volume of space; and, optionally or preferably, wherein the housing comprises a mechanism configured to alter the volume of the housing between a first volume and a second volume and, optionally, the second volume is greater than the first volume and the mechanism is configured to reversibly increase the volume of the housing.

27. The inhaler housing of claim 24, wherein at least one flow directing element is disposed at or adjacent the locating portion; and/or wherein at least one flow directing element is disposed adjacent the outlet, and optionally is disposed on or near the mouthpiece.

28. The inhaler housing of claim 24, wherein at least one flow directing element is disposed adjacent the outlet, and optionally is disposed on or near the mouthpiece, and wherein the at least one flow directing element disposed adjacent the locating portion and the at least one flow directing element disposed adjacent the outlet form or define a primary flow path for aerosolized medication through the space enclosed by the housing.

29. The inhaler housing of claim 24, wherein: the mouthpiece is securable or secured to the housing.

30. The inhaler housing of claim 24, wherein the mouthpiece is moveably secured to the housing; and the mouthpiece is moveable between a first position in which the outlet is open and a second position in which the outlet is closed; and, optionally or preferably,
wherein the housing is substantially sealed when the mouthpiece is in the second position, and optionally wherein the mouthpiece is substantially flush with an outer surface of the housing when the mouthpiece is in the second position; and/or
wherein mouthpiece and/or the housing is configured to facilitate moving the mouthpiece from the second position to the first position; and, optionally, wherein an outer surface of the housing comprises a notch or recess configured to enable a user to move the mouthpiece from the second position to the first position, and vice versa, and/or wherein the mouthpiece is shaped or comprises a projection to enable a user to move the mouthpiece from the second position to the first position, and vice versa.

31. The inhaler housing of claim 29, wherein the at least one flow directing element disposed adjacent the outlet is configured to direct a flow of aerosolized medication when the mouthpiece is in the first position.

32. The inhaler housing of claim 24, further comprising a valve located in or in the vicinity of the mouthpiece and, optionally or preferably, where the valve is an inspiratory valve.

33. The inhaler housing of claim 24, wherein the housing comprises a plurality of housing portions, wherein the plurality of housing portions are attachable to and detachable from one another; and, optionally or preferably wherein the housing portions comprise (a) an upper part and a lower part, (b) a first side part and a second side part or (c) a front part and a back part.

34. The inhaler housing of claim 33, wherein the plurality of housing portions are configured to be removably attached via at least one of corresponding male and female engagement features, a press fit or friction fit, corresponding screw threads and/or one or more resilient elastic clips.

35. The inhaler housing of claim 24, wherein the at least one flow directing element disposed within the housing is arranged at an angle of between:
i) substantially 110° and substantially 130° relative to an initial direction of flow of aerosolized medication, or substantially 85° and substantially 135° relative to an initial direction of flow of aerosolized medication, or substantially 95° and substantially 130° relative to an initial direction of flow of aerosolized medication, or substantially 105° and substantially 125° relative to an initial direction of flow of aerosolized medication, or substantially 110° and substantially 120° relative to an initial direction of flow of aerosolized medication or substantially 115° relative to an initial direction of flow of aerosolized medication; and/or
ii) substantially 50° and substantially 70° relative to an internal surface of the housing, or substantially 15° and substantially 45° relative to an internal surface of the housing, or substantially 18° and substantially 40° relative to an internal surface of the housing, or substantially 20° and substantially 35° relative to an internal surface of the housing, or substantially 22° and substantially 30° relative to an internal surface of the housing or substantially 25° relative to an internal surface of the housing.

36. The inhaler housing of claim 24, wherein the at least one flow directing element disposed within the housing is or comprises one or more of a flat surface or plate and a curved surface or plate.

37. The inhaler housing of claim 24, further comprising a whistle disposed in a wall of the housing.

38. The inhaler housing of claim 24, further comprising a cap receivable in and/or attachable to the locating portion, wherein the cap is configured to cover said canister when it is received in the locating portion, and optionally wherein the cap is flush with an outer surface of the housing when received in/attached to the locating portion.

39. The inhaler housing of claim 24, further comprising one or a plurality of expiratory vents or holes and, optionally or preferably, wherein the expiratory vents or holes are provided in a surface of the housing in the vicinity of or under the mouthpiece; and/or optionally or preferably, wherein the plurality of expiratory vents or holes are provided in regular or irregular pattern, a grid or a plurality of rows each comprising one or a plurality of expiratory vents or holes.

40. The inhaler housing of claim 24, wherein the housing is substantially triangular, square or rectangular in lateral cross-section.

41. An inhaler comprising:
a housing enclosing a space; and
an aerosolized medication canister;
wherein the housing comprises:
a locating portion configured to receive the canister;
an outlet comprising a mouthpiece;
at least one flow directing element disposed within the housing;
wherein the at least one flow directing element is configured to direct a flow of aerosolized medication toward the outlet;
wherein the space enclosed by the housing is configured to reduce a velocity of the aerosolized medication prior to the aerosolized medication reaching the outlet for inhalation by a user.

42. The inhaler of claim 41, wherein the canister is a metered-dose inhaler canister.

43. The inhaler of claim 41, wherein the canister is removable from the locating portion.
